(19) United States
(12) Patent Application Publication
(10) Pub. No.: US 2011/0065944 A1
(43) Pub. Date: Mar. 17, 2011

(54) NEW REACTIONS OF FULLERENES

(75) Inventor: Darren K. Macfarland, Danville, VA (US)

(73) Assignee: Luna Innovations Incorporated`, Roanoke, VA (US)

(21) Appl. No.: 12/992,746

(22) PCT Filed: Oct. 22, 2008
(86) PCT No.: PCT/US08/11997

§ 371 Date: Nov. 15, 2010

Related U.S. Application Data
(60) Provisional application No. 61/071,756, filed on May 15, 2008.

(51) Int. Cl. C07F 5/00; C07C 211/33; B82Y 40/00

(52) U.S. Cl. 556/1; 564/461; 977/734

(57) ABSTRACT

A process for producing a fullerene derivative. The process comprises reacting a fullerene compound with a primary amine in the presence of a peroxide.

### FIELD

This disclosure relates to new reactions of fullerenes with primary amines in the presence of a peroxide.

### BACKGROUND

Fullerenes are a third allotrope of carbon. Unsubstituted fullerenes are insoluble in water and soluble only in a small number of aromatic solvents. Some fullerene derivatives have been found to exhibit biological activities. For instance, certain endohedral metallofullerenes have been found to be suitable as magnetic resonance imaging (MRI) contrast agents. However, due to the limited solubility, the applications of the fullerene derivatives are still restricted, particularly, as biological tools.

### SUMMARY

An exemplary embodiment is directed to a process for producing a fullerene derivative, comprising reacting a fullerene compound with a primary amine in the presence of a peroxide, to form the fullerene derivative.

The present disclosure describes a convenient process producing fullerene derivatives with improved solubility, which makes it possible to use these fullerene derivatives as MRI contrast agents, fullerene therapeutics, etc.

### BRIEF DESCRIPTION OF THE DRAWINGS

FIG. 1 illustrates a reaction scheme of C₆₀ with a secondary amine.

FIG. 2 shows the reaction products of t-butyl peroxide radical with C₆₀.

FIG. 3 illustrates a reaction scheme of fullerene peroxide with nucleophile NaOCH₃.

FIG. 4 shows crystal structures of fullerene radical addition products.

FIG. 5 illustrates successive addition scheme of radical R to a fullerene structure.

FIG. 6 shows a mass spectrum of C₆₀-perfluorohexyl radical addition product.

FIG. 7 illustrates a reaction scheme of radical addition to fullerene C₆₀ via copper ion.

FIG. 8 shows an X-ray crystal structure of C₆₀(CF₃)₁₂.

FIGS. 9(a) and (b) show stick models of Gd₃N@C₈₀ structures.

FIG. 10 illustrates a reaction scheme of radical addition of CF₃I to Sc₃N@C₈₀.

FIG. 11 is a photograph of the gel electrophoresis data of C₇₀-monoethyleneglycol amine adduct (C₇₀-MEG), Hydrochalarone-1 (Gd₃N@C₈₀-MEG) and Hydrochalarone-3 (Gd₃N@C₈₀-TEG).

FIGS. 12(a) and (b) show IR spectra of C₇₀-MEG before and after D₂O addition.

FIGS. 13(a) and (b) show ¹H NMR spectra of MEG and undialyzed C₇₀-MEG in D₂O.

FIG. 14 shows the elemental analysis of Hydrochalarone-3.

FIGS. 15 (a) and (b) show IR spectra of C₇₀-MEG and Hydrochalarone-3.

FIGS. 16 (a) and (b) show IR spectra of Hydrochalarone-1 and Hydrochalarone-3.

FIG. 17 shows UV/Vis spectrum of Hydrochalarone-6 in water.

FIGS. 18(a) and (b) show IR spectra of GdTMS-decylamine and Hydrochalarone-1.

FIGS. 19 (a) and (b) show ¹H NMR spectra of MEG-TFA in D₂O and YTMS pre-dialysis.

FIG. 20 shows ¹H NMR spectrum of post-dialysis Y Hydrochalarone-1 in D₂O.

FIGS. 21 (a) and (b) show TGA spectra of Hydrochalarone-6 and unfunctionalized Gd₃N@C₈₀.

### DETAILED DESCRIPTION OF EMBODIMENTS

Various studies have been conducted on water-solubilization of fullerenes. The addition of polar groups to the fullerene to render the molecule water-soluble was reported in a study by Friedman. Friedman, S. H., et al., Inhibition of the HIV-1 Protease by Fullerene Derivatives: Model Building Studies and Experimental Verification, J. Am. Chem. Soc. (1993) 115, 6506-6509. In another example, the addition of hydroxyl groups was described by Chiang who reacted C₆₀ with aqueous acids to provide a polyhydroxyl fullerene compound. Chiang, L. Y., et al. Multi-hydroxy Additions onto C₆₀ Fullerene Molecules J. Chem. Soc. Chem. Comm. (1992) 1791.

An early review of other methods was reported by Nakamura: Yomago, S., Nakamura, E. et al. In vivo Biological Behavior of a Water-miscible Fullerene-C14 Labeling, Absorption, Distribution, Excretion, and Acute Toxicity Chemistry and Biology, (1995), 2(6), 385-389. Lamparth et al. described the synthesis of C₆₀ malonic acids by the reaction of fullerene with diethyl bromomalonate, followed by saponification of the formed malonates in the presence of NaH. The resulting C₆₀ malonic acids are soluble in basic to neutral water and insoluble in aqueous acids. Lamparth et al., Water-soluble Malonic Acid Derivatives of C₆₀ with a Defined Three-dimensional Structure, J. Chem. Soc., Chem. Commun. 1994, 1727-28. Further, Sawamura et al. reported the preparation of water soluble anionic fullerenes, i.e., pentaaryl and pentamethylfullerene anions R₅C₆₀⁻ by the reaction of fullerene with an organocopper reagent. Sawamura et al., Pentaorgano[60]fullerene R₅C₆₀⁻. A Water Soluble Hydrocarbon Anion, Chem. Lett. 2000, 1098-99.

Addition reactions of aliphatic amines to fullerenes have also been explored. It was found that some primary and secondary amines add readily and repeatedly across C₆₀ fullerene producing complex reaction mixtures composed of numerous structures and isomers. Eudl et al., Fullerenes: Synthesis, Properties and Chemistry of Large Carbon Clusters, Ed.: G. S. Hammond and V. J. Kuck, ACS Symp. Ser. 1992, 48 (11), 161-175. Other studies showed that certain secondary amines, such as N,N′-dimethylethylenediamine, piperazine and morpholine, can result in well-defined addition products. Kampe et al., Diamino and Tetramino Derivatives of Buckminsterfullerene C₆₀, Angew. Chem., Int. Ed. Engl. 1992, 32, 1174-76; Schick et al., Reaction of [60]fullerene with morpholine and piperidine: preferred 1,4-additions and fullerene dimer formation, J. Chem. Soc., Chem. Commun. 1995, 2023-24.

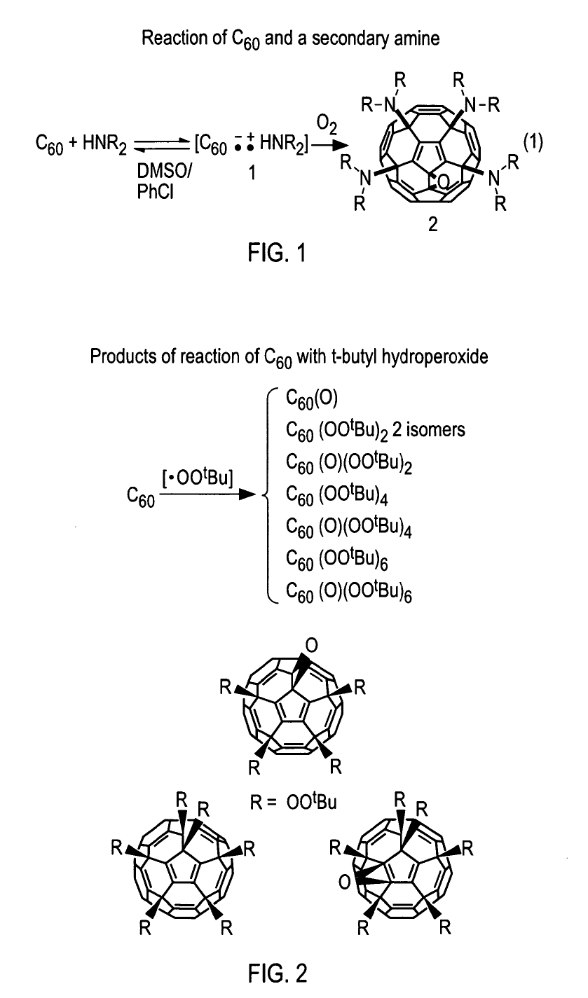

Amine additions to fullerenes are well known and have been reviewed. See e.g. Miller, G P Reactions between aliphatic amines and [60]fullerene: a review C. R. Chemie 9, 952-959 (2006). In addition, the reaction of secondary amines (HNR₂) with fullerenes catalyzed by molecular oxygen in air has been reported (FIG. 1). See e.g. Nakamura, Regioselective Oxygenative Tetraamination of [60]Fullerene. Fullerene-mediated Reduction of Molecular Oxygen by Amine via Ground State Single Electron Transfer in Dimethyl Sulfoxide, J. Org. Chem., 70, 4826-4832 (2005). Nakamura states that “ . . . primary amines give no isolable products” in this reaction.

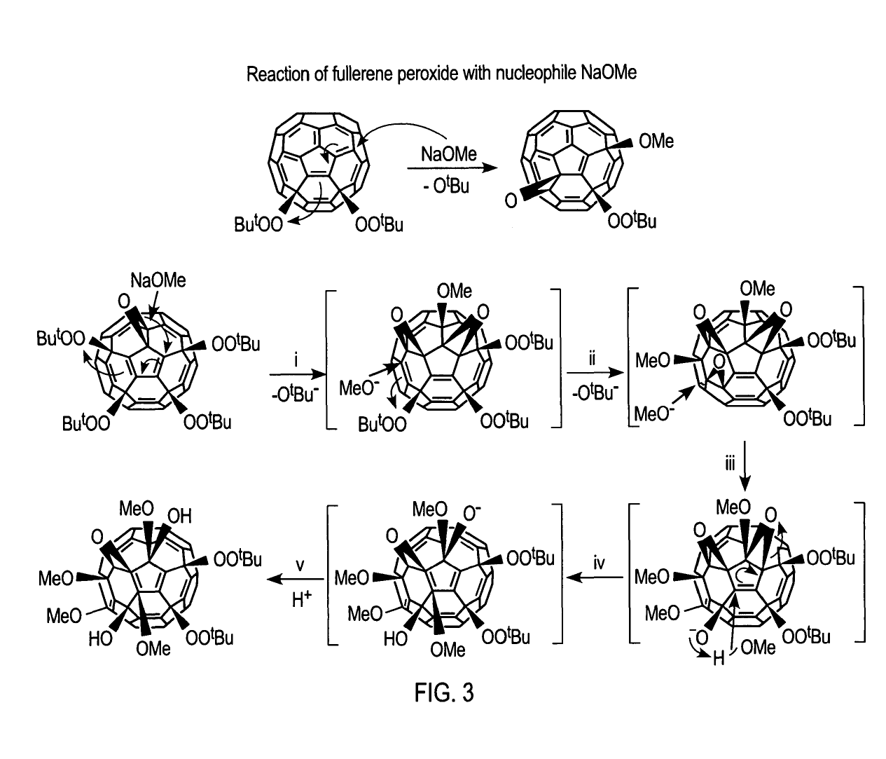

A similar pattern addition of organic peroxides such as tert-butyl hydroperoxide has been extensively studied by Gan (FIG. 2). See e.g. Gan, Fullerene peroxides, C R Chimie, 9, 1001-1004 (2006). Gan comments that “nucleophiles react with fullerene peroxides according to either SN1 or SN2′ or SN2″ pathway.” The only example described therein is the addition of CH₃O⁻ (methoxide) that displaces a peroxide to give one or more adducts. The assigned structures and pathways are shown in FIG. 3.

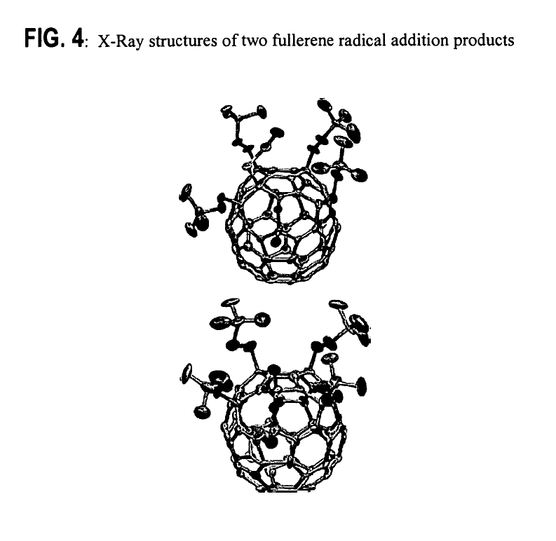

Yang reported the opening of holes in fullerene using peroxides. Two cage-open fullerene derivatives 2 and 4 gave X-ray crystal structures as shown in FIG. 4. Yang, et al., Controlled region-and chemoselective addition of isothiocyanate to the dione moiety of a cage-opened fullerene-mixed peroxide derivative, Chem. Comm. 1980-1982 (2008).

More recently, Nakamura reported the addition reaction of secondary amines to C₆₀ which was catalyzed by cumene hydroperoxide. Nakamura et al., Synthesis of Oxy Aminated [60] and [70] Fullerenes with Cumene Hydroperoxide as Oxidant, Chem. Lett. 36, 20-21 (2007).

Based on Nakamura's work, all of these addition processes require that the fullerene first reacts with the amine to form a charge transfer complex, which is then oxidized by the molecular oxygen or peroxide.

The present inventor has found a new reaction that secondary amines do not participate in. Furthermore, neither molecular oxygen nor cumene hydroperoxide affects the reaction. In particular, the reaction proceeds with a specifically preformed and reactive amine radical. For example, a reaction involving aminopropyl-surface-modified silica as the amine was performed. Butanone peroxide was added to the silica-bound amine, then washed off through filtering and rinsing of the silica. This activated silica reacted readily with a number of fullerenes in solution when a mixed-fullerene solution was added. These data suggest that the key step in the reaction is the formation of a novel nitrogen-centered radical.

The terms “fullerene” and “fullerene compound,” as used herein, denote a compound containing a fullerene cage network, and include both an empty fullerene compound with no additional atoms within its cage network and an endohedral metallofullerene. The term “endohedral metallofullerene,” as used herein, denotes a fullerene compound where one or more metal atoms are encapsulated inside a fullerene cage network. Accepted symbols for elements and subscripts to denote numbers of elements are used herein. Generally, all elements to the right of an @ symbol are part of the fullerene cage network, while all elements listed to the left of the @ symbol are contained within the fullerene cage. For example, under the notation Gd₃N@C₈₀, the Gd₃N trimetallic nitride is situated within a C₈₀ fullerene cage.

The term “TRIMETASPHERE®” (TMS) denotes a member of a family of endohedral metallofullerenes which contain a trimetallic nitride compound in the fullerene cage. One example Gd₃N@C₈₀ is referred to as GdTMS. Some examples of TRIMETASPHERE® are discussed in U.S. Pat. Nos. 6,303,760 and 6,471,942, and U.S. Patent Application Publication No. 2004/0054151.

The term “fullerene derivative,” as used herein, denotes a fullerene-containing product obtained in the reaction described herein.

The present disclosure describes new reactions of fullerene compounds with amines in the presence of a peroxide.

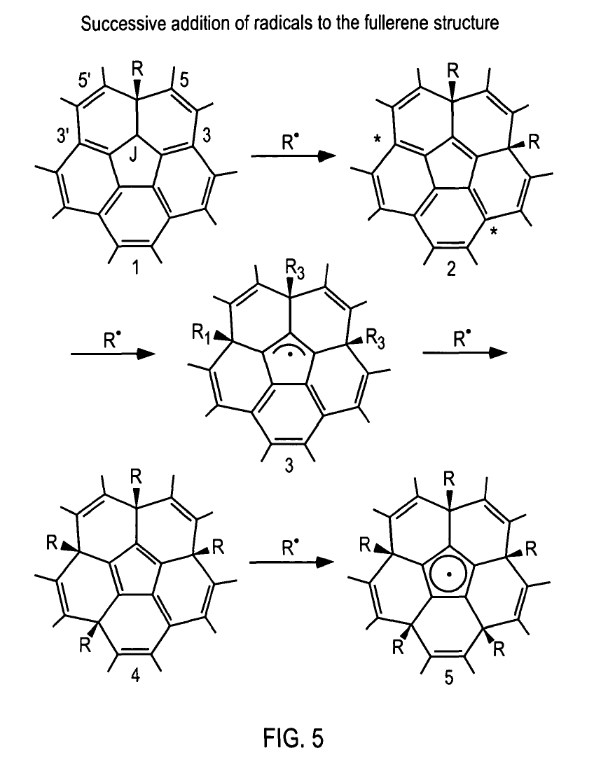

Soon after the discovery of fullerene C₆₀, its chemistry as a “radical sponge” was recognized and extensively studied. C₆₀ is a highly conjugated olefin with carbon-carbon bonds that can react avidly with radicals to produce increasingly more stable allylic radicals and eventually multiple adducts. The unique feature of all these fullerenes is the presence of six 5-membered rings (each closed fullerene structure must have six five membered rings) that can accept five radical (R.) groups to produce the very stable cyclopentadienyl radical (FIG. 5). See e.g. Krusic et al., Radical Reactions of C₆₀, Science, 254, 1183-85 (1991).

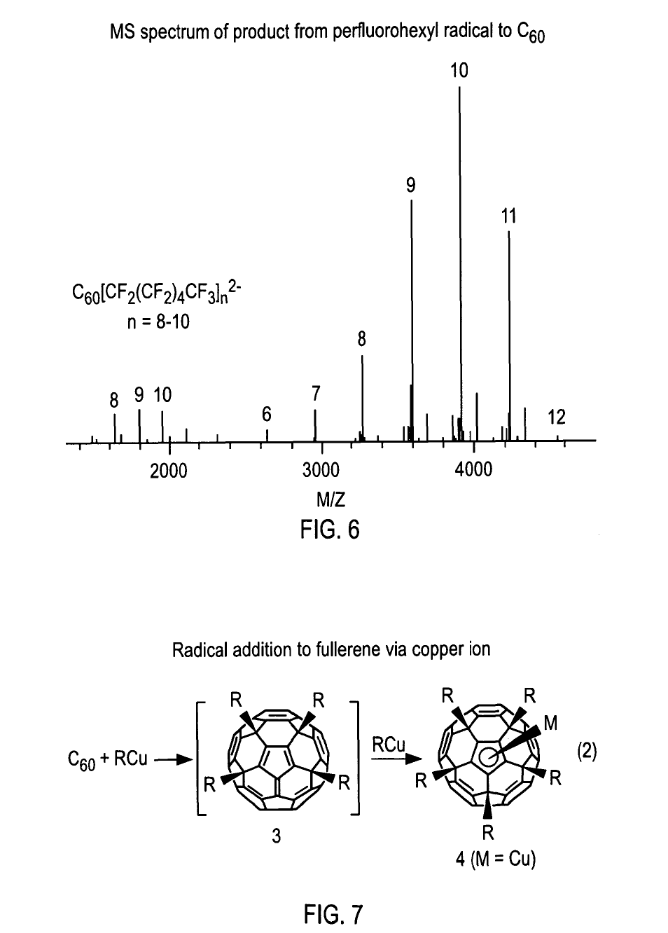

The addition of alkyl radicals to fullerenes was extensively investigated by the Dupont group. For example, heating perfluorohexyl iodine with C₆₀ at 178° C. gave addition of 8-11 groups to the sphere (FIG. 6). Fagan, P J et al., Production of Perfluoroalkylated Nanospheres from Buckminsterfullerene, Science, 262, 404-407 (1993).

More recently, the controlled addition of radicals to fullerenes has been adapted as a reliable process using radical-producing reagents such as organocuprates (FIG. 7). Matsuo, Y. and Nakamura, N., Selective Multiaddition of Organocopper Reagents to Fullerenes Chem. Rev., 108, 3016-3028 (2008).

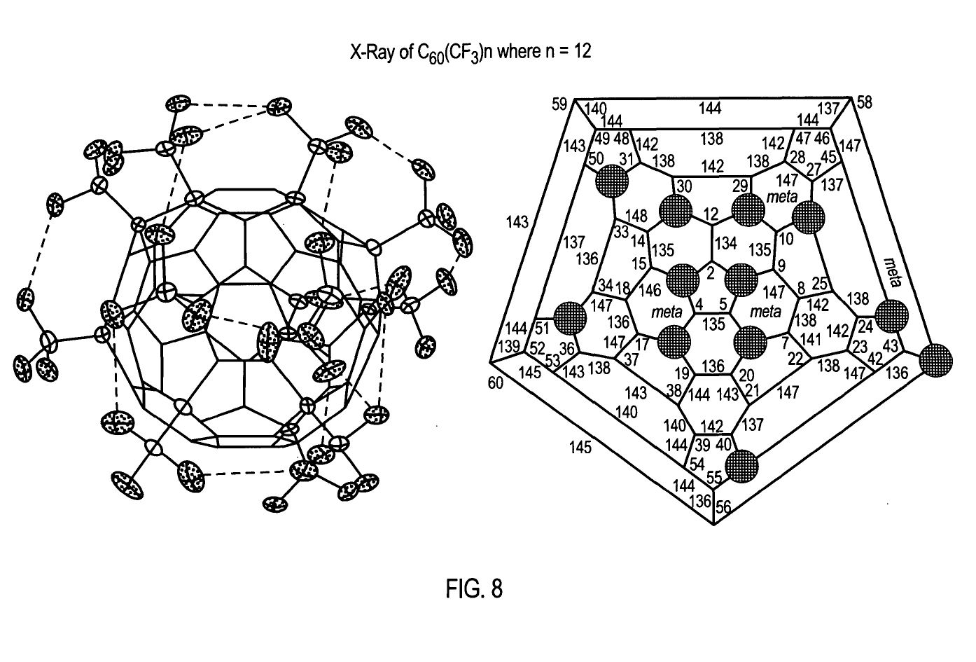

While much of the regiochemistry of radical fullerene additions takes place at the unique fullerene pyracylene bonding patterns of interconnecting carbons, another pathway involves 1,4-addition on benzene rings. For example, Kareev has reported successive CF₃.radical additions to C₆₀ which result in a product with adducts that was completely characterized by X-ray crystallography (FIG. 8). See Kareev et al., X-ray structure and DFT study of C₁-C₆₀(CF₃)₁₂. A high-energy, kinetically-stable isomer prepared at 500° C., Chem. Commun., 2007, 1650-1651

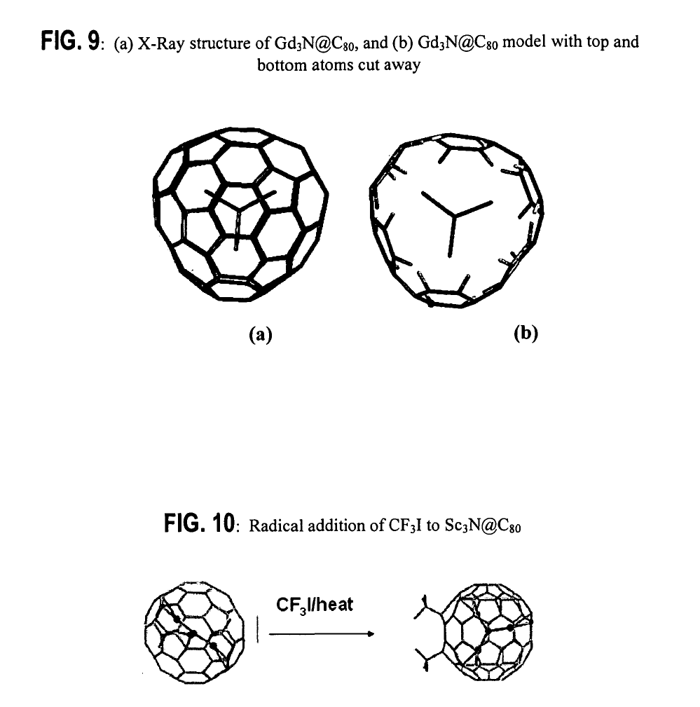

Addition chemistry of TRIMETASPHERE® (“TMS”) C₈₀ compounds is often different from empty cage fullerenes, since the fullerene pyracylene bonding pattern is not present in C₈₀. There are, however, benzene rings that can participate in 1,4-addition reactions. In fact, if one examines the distribution of benzene rings on the surface of a TMS, a “belt” of 12 rings are arrayed in a biphenyl-type linkage. Every other benzene ring is situated over a Gd metal atom (FIG. 9) and indeed appears to participate in η6-bonding arrangements. FIG. 9(a) is an X-Ray structure of Gd₃N@C₈₀, and FIG. 9(b) is a Gd₃N@C₈₀ model with top and bottom atoms cut away to reveal arrangement of the internal trimetallic nitride and surrounding belt of benzene rings.

The addition of radicals to TMS compounds takes place via 1,4-addition on benzene rings around this “biphenyl-belt”. Shustova has recently reported the addition of trifluoromethyl radical to Sc₃N@C₈₀. While the product mixture contains mainly a series of Sc₃N@C₈₀(CF₃)ₙ where n=8-12, they were able to isolate and characterize the bis adduct as shown in FIG. 10. Note that the addition preferentially occurred in a 1,4-pattern on the benzene ring in between two η6-bonded scandium atoms (FIG. 10). Shustova, N, B. et al, Radical Trifluoromethylation of Sc₃N@ C₈₀, J. Am. Chem. Soc. 129 (38), 11676-11677 (2007).

### Fullerene Compounds

The fullerene compound described herein may contain a fullerene cage represented by Cₘ. m represents an integer, in particular, an even value, from about 60 to about 200. In one embodiment, m is 60, 68, 70, 74, 78, 80, 82, 90, 92, and 94. Further, m is preferably an integer from about 60 to about 88. The fullerene cage may optionally have one or more substituents. The substituents on the fullerene cage are not particularly limited, so long as they do not interfere with the reaction described herein.

When an endohedral metallofullerene is used as the fullerene compound, the metal atom(s) encapsulated inside a fullerene cage network are not particularly limited. When multiple atoms are encapsulated inside a fullerene cage network, they may be the same or different. In one embodiment, the endohedral metallofullerene contains at least one paramagnetic metal and particularly, an element having one or more unpaired electrons. In a preferred embodiment, a rare earth element, a group IIIB element in the periodic table of elements or the like can be used as the encapsulated atom. Examples of suitable rare earth elements and group IIIB elements may include, but are not limited to, gadolinium (Gd), scandium (Sc), erbium (Er), holmium (Ho), yttrium (Y), lanthanum (La), thulium (Tm), dysprosium (Dy), terbium (Tb) and ytterbium (Yb).

In one embodiment, the fullerene comprises C₆₀, C₇₀, or Gd₃N@C₈₀. It was found that C₆₀ and C₇₀ react about 4-fold faster than Gd₃N@C₈₀ under the same reaction conditions.

### Amines

The amines which are suitable for the reaction described herein can be quite diverse and include ammonium, alkyl amines, amino acids, poly ether amines, etc. Hydroxyl group-containing amines are generally not preferred due to potential oxidation reactions of the hydroxyl group by the peroxide. In an embodiment, the amine group in the amine is attached to a primary or secondary carbon atom. Preferably, such amine group is attached to a primary carbon atom.

In an embodiment, the amine described herein comprises a primary amine. Examples of suitable primary amine compounds may include compounds represented by RNH₂. In one embodiment, R may represent H, NH₂(CH₂)ₐCH₃ and NH₂(CH₂CH₂O)ₐCH₃ wherein a is an integer of 1 or more, and preferably, 1 to 20. In an embodiment, a is 1, 3, 5 or 11. Specifically, the primary amine compounds may be represented by the formula RNH₂ wherein R is H, ethyl, propyl, hexyl, dodecyl, —CH(CH₂OCH₃)₂, —(CH₂)₄—NH₂, —(CH₂)₁₀—NHBoc, —(CH₂)₂—COOH, —CH(COOCH₂CH₃)(CH₂)₂Ph, —CH₂-18-crown-6, -glyme-OCH₃, -glyme-NHBoc, -triglyme-OCH₃, -triglyme-NHBoc, -hexaglyme-OCH₃ and -hexaglyme-NHBoc (glyme refers to a repeating ethylene glycol unit).

### Peroxides

The peroxide described herein preferably contains butanone peroxide. Other suitable peroxides, such as cumene hydroperoxide, can also be used. Moreover, when water is used as the solvent for the reaction described herein, hydrogen peroxide may be used as the peroxide.

### Solvents

The solvent for this reaction may comprise an organic solvent, such as toluene, xylene, benzene and ortho-dichlorobenzene. In addition, certain partially reacted fullerene products have a reasonable solubility in water, and can be further reacted in water. These solvents may be used individually or in combination thereof. In an embodiment, the solvent contains xylene.

### Reactions of Fullerene Compounds

The present inventor has discovered a new reaction type that provides a favorable route to functionalization of fullerene compounds. The reaction is based on a unique activated nitrogen-centered radical species generated from an amine and a peroxide.

The term “Hydrochalarone,” as used herein, refers to the class of compounds resulting from the reaction of the fullerenes, peroxide and amine. Hydrochalarone-1, Hydrochalarone-3 and Hydrochalarone-6 refer to the compounds obtained by using, as the amine, monoethylene glycol (MEG), triethylene glycol, and hexaethylene glycol, respectively.

Although not wishing to be bound by any theory, it is believed that the reaction of a peroxide, such as butanone peroxide, with an amine, such as a primary amine, leads to formation of an RN(OH). reactive intermediate that has stability of at least several minutes but not many hours. In the presence of a fullerene compound, the RN(OH). reacts rapidly with the benzene rings on the fullerene cage, forming a fullerene radical, which, in turn, may react with other radical or non-radical species present in the reaction mixture.

The resulting substituted fullerene molecules may be repeatedly reacted with the existing radical species, thereby forming multi-substituted fullerene molecules, polymers or both. Further substitutions may occur at any available position of the substituted fullerene, e.g., a carbon atom on the fullerene cage or any site on the substituent thereof.

In such a reaction, the concentration of the fullerene can range from about 0.25 mg/mL to about 5.0 mg/mL, based on the volume of the solvent used. In an embodiment, the fullerene can be used in a concentration of about 1 mg/mL to about 2 mg/mL, based on the volume of the solvent.

Generally, the amine compound and the peroxide can be used in large excess of the fullerene compound. In an embodiment, the fullerene:amine molar ratio can range from about 1:50 to about 1:750 and preferably, about 1:500. The amine:peroxide molar ratio can range from about 1:1 to about 1:10, preferably, about 1:5, and more preferably, about 1:3. It should be noted that butanone peroxide contains three peroxide units. In a particular embodiment, the fullerene:amine: effective peroxide unit molar ratio is about 1:500:1000. When water is used as the solvent, the amounts of the amine compound and the peroxide may be relatively higher.

The reaction of the fullerene compound may be conducted at ambient conditions and the reaction proceeds at a varied rate depending on the nature of fullerene, amine, peroxide and/or solvent. The reaction process can be monitored by the formation of precipitate of the product from the reaction mixture.

The reaction temperature may be raised to accelerate the reaction rate, if necessary or desired. In this case, the reaction temperature is preferably set below the boiling point of the amine and/or solvent used therein. In an embodiment, the reaction can be carried out at ambient temperature to about 80° C.

When a C₈₀ TRIMETASPHERE® is used, the reaction is preferably run at 75° C. On the other hand, when a C₆₀ or C₇₀ TRIMETASPHERE® is used, the reaction runs relatively fast at ambient temperature and does not need heating. When using Gd₃N@C₈₀, the reaction yield can be determined by ashing a sample of a known quantity of the product and determining the Gd₂O₃ concentration therein. The Gd₂O₃ concentration can be determined by measuring relaxivity and comparing the measured value to a previously determined concentration/relaxivity calibration curve.

When an empty fullerene is used, the reaction is substantially complete in about 10 minutes to about 60 minutes at ambient temperature. When Gd₃N@C₈₀ is used, the reaction is substantially complete in about 10 minutes to about 60 minutes at 75° C. and from about 1 hour to about 16 hours at ambient temperature. The isolated reaction yields of Gd₃N@C₈₀ can range from about 60% to about 80%, with the remainder being aggregated partially reacted materials. The crude reaction products may be purified by various methods, such as precipitation, dialysis and column chromatography.

In one specific reaction of NH₂(CH₂CH₂O)₃CH₃ with Gd₃N@Cₘ in the presence of butanone peroxide, mono- and/or multi-substituted fullerene derivatives may be formed. It is possible that the resulting reaction products may contain, in addition to the —N(OH)R addition product(s), the —ONHR addition product(s), the —NHR addition product(s) or a mixture of two or more of these products. It is also possible that the intermediate fullerene radicals polymerize, thereby forming one or more polymers in the reaction mixture.

Further, in the reaction mixture, the number of substituents on each fullerene cage may vary, depending, to some extent, on the nature of fullerene, substituents and reaction conditions. Thus, it is likely that any particular reaction may generate a mixture of fullerene derivatives having different types and/or numbers of substituents on the fullerene cages thereof.

When a fullerene derivative having more than one types of substituents is desired, different amines may be employed simultaneously (“one-pot reaction”) or in sequence, which may vary and can be optimized by those skilled in the art.

The fullerene derivatives obtained in the reaction described herein may have a relatively high solubility in water and/or other organic solvents, such as ether. Generally, an amine having fewer hydrocarbon groups and/or one or more heteroatoms, such as oxygen and nitrogen, tends to result in a fullerene derivative having a relatively higher water solubility. On the other hand, an amine having more hydrocarbon groups tends to result in a fullerene derivative having improved ether solubility. For instance, the solubility of Gd₃N@ C₈₀/NH₂CH₂CH₂OCH₃ adduct (“Hydrochalarone-1”) in water is about 100 mg/mL. This figure is roughly two orders of magnitude above the highest values for the known fullerenes and their derivatives. In an embodiment, the fullerene derivative described herein has a water solubility of greater than about 40 mg/mL. Further, the solubility of Gd₃N@C₈₀/NH₂(CH₂)₁₁CH₃ adduct in ether is greater than about 10 mg/mL. Depending on the end uses, it is possible to fine tune the water/ether solubility of the fullerene derivatives described herein by employing a suitable amine in the reaction.

The improved solubility makes the fullerene derivatives described herein suitable for various applications. Particularly, the improved water solubility allows the fullerene derivatives described herein to be compatible with biological systems and thus useful in the biological fields. In addition, the endohedral metallofullerene derivatives described herein preferably have a relaxivity of greater than about 40 mM⁻¹s⁻¹, which makes them potentially useful as MRI contrast agents.

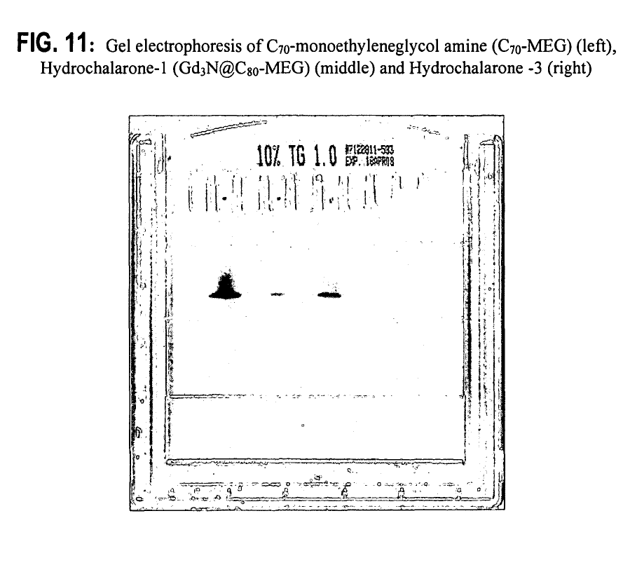

The increased solubility is believed to be attributable to the attachments on the fullerene cage. In addition, a charge residing on the cage may also be accountable for the increased water solubility. For example, gel electrophoresis experiments show that Gd₃N@C₈₀/NH₂(CH₂CH₂O)₃CH₃ adduct (“Hydrochalarone-3”) bears a negative charge (FIG. 11). In one embodiment, the endohedral metallofullerene compound may have one or more negative charges and counter cation(s). The counter cation may be any positively charged species present in the medium. Specific examples of the cations may include, but are not limited to, H⁺ and protonated amine reagent used in the reaction, e.g., protonated MEG.

Specifically, FIG. 11 shows gel electrophoresis results of C₇₀-monoethyleneglycol amine (C₇₀-MEG) (left), Hydrochalarone-1 (Gd₃N@C₈₀-MEG) (middle) and Hydrochalarone-3 (Gd₃N@C₈₀-TEG) (right) moving toward cathode (bottom). The gel electrophoresis was conducted according to Novex® Tris-Glycine polyacrylamide gel chemistry (Novex® 10% Tris-Glycine Gel 1.0 mm, 10 well). Novex® Tris-Glycine polyacrylamide gel chemistry is based on the Laemmli system (1) with minor modifications for maximum performance in the pre-cast format. These gels do not contain SDS and can therefore be used to accurately separate both native and denatured proteins. Novex® Tris-Glycine Gels are made with high-purity, strictly quality-controlled reagents: Tris base, HCl, acrylamide, bisacrylamide, TEMED, APS, and highly purified water. They do not contain SDS. Tris-Glycine Running Buffer without SDS is used as buffer. The testing sample is prepared by combining 25 uL of each sample with 5 uL of glycerol per well. The current is loaded @ 50 Volts (20 minutes) and then increased to 120 Volts (30 minutes) once loaded.

The present invention is further illustrated by the following specific examples but is not limited hereto.

### EXAMPLES

Unless specified, all the commercially available materials were used herein without further purification. All the measurements including weight and temperature were uncorrected.

### Example 1

### Reaction of an Empty Fullerene with NH₂CH₂CH₂OCH₃

C₆₀ (15 mg) was dissolved in xylene (10 mL) by means of sonication for 30 seconds. To this solution were added NH₂CH₂CH₂OCH₃ (300 mg) and butanone peroxide (750 mg of a 31% commercially available solution in 2,2,4-trimethyl-1,3-pentanediol diisobutyrate). The mixture was shaken at ambient temperature for 1 hour. During the course of the reaction, the solution turned from purple to yellow and a brown precipitate formed. The solution was decanted leaving the brown oil. The oil was washed with toluene (5 mL×2) and ether (5 mL×2), then dissolved in water (10 mL) and filtered through a 100 nm filter, yielding the product in solution. The resulting solution may be further purified by dialysis using 1000 MWCO tubing. Alternatively, the product may be precipitated with isopropanol from a methanol solution to yield a brown powder.

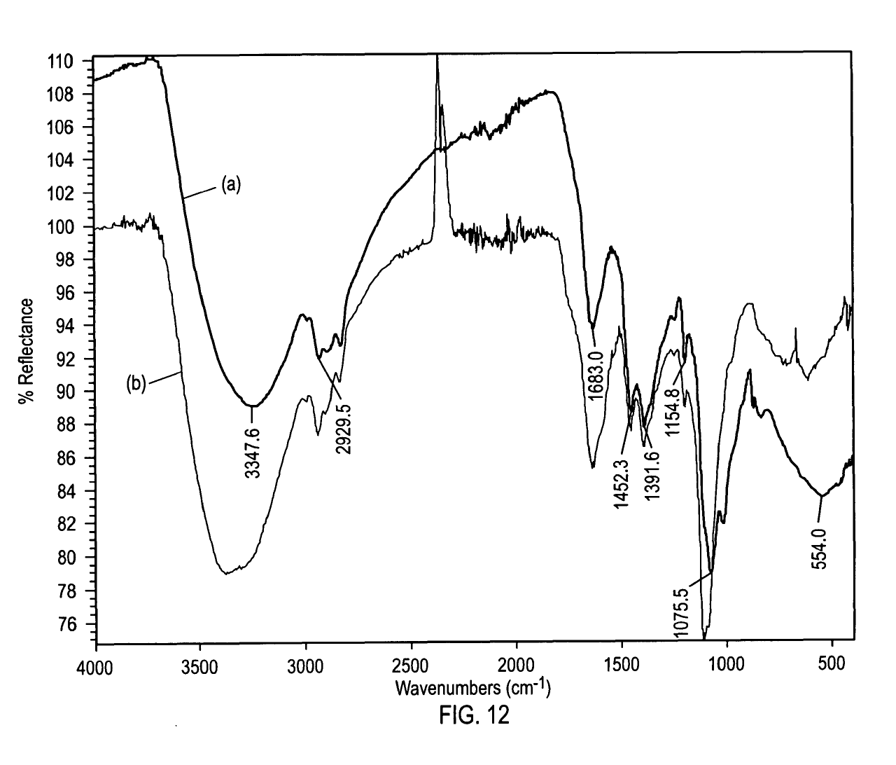

The same reaction was also conducted by substituting C₇₀ for C₆₀. IR spectra of C₇₀-monoethyleneglycol amine (C₇₀-MEG) before addition of D₂O and after heating in D₂O (FIGS. 12(a) and (b), respectively) show lack of H/D exchange with heating treatment.

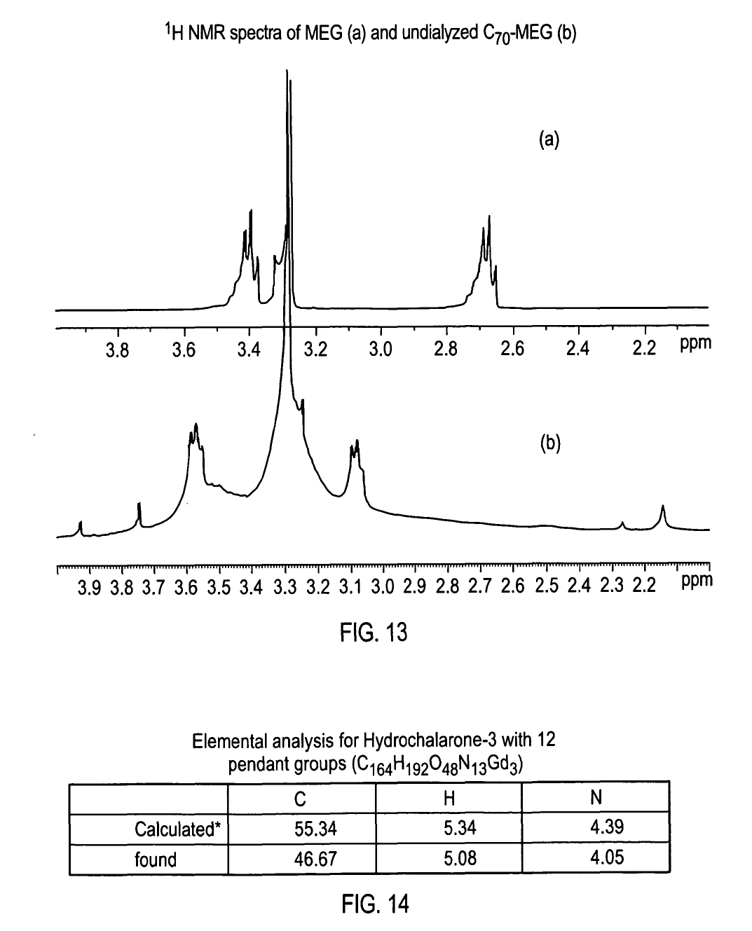

Further, ¹H NMR spectra of MEG (FIG. 13(a)) and undialyzed C₇₀-MEG (FIG. 13 (b)) show the presence of MEG-H⁺ cation with C₇₀-MEG in the reaction mixture.

### Example 2

### Reaction of an Empty Fullerene with Decyl Amine NH₂(CH₂)₁₁CH₃

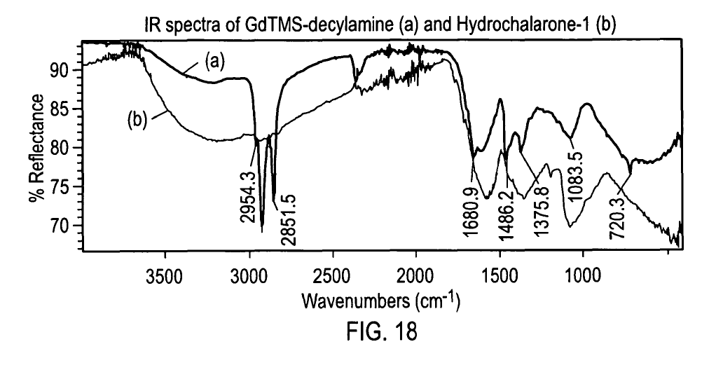

To a solution of C₆₀ (36 mg) in xylene (20 mL) were added NH₂(CH₂)₁₁CH₃ (2.1 g) and butanone peroxide (4.2 g of a 31% commercially available solution in 2,2,4-trimethyl-1,3-pentanediol diisobutyrate). The reaction was observed by a rapid color change to yellow-brown. The crude product was purified by silica chromatography, using diethyl ether as eluant. The obtained product was an ether-soluble dark brown oil (see FIG. 18(a)).

### Example 3

### Reaction of an Endohedral Metallofullerene with NH₂(CH₂CH₂O)₃CH₃

Gd₃N@C_{g}0 (10 mg), NH₂(CH₂CH₂O)₃CH₃ (250 mg) and butanone peroxide (1500 mg of a 31% commercially available solution in 2,2,4-trimethyl-1,3-pentanediol diisobutyrate) were mixed with xylene (10 mL). The resulting mixture was agitated at 75° C. for 1 to 2 hours. At this time, the reaction mixture became nearly colorless but with precipitate. The colorless solvent was decanted and the remaining oil was washed with toluene and ether. The oil was then dissolved in water. The aqueous extract was passed through a Sephadex column twice (eluting at the solvent front) and/or dialyzed with 1000 MWCO tubing. Rotavapping the aqueous solution can be difficult; however, warming under a steady nitrogen stream yielded a black powdery product (“Hydrochalarone-3”). The relaxivity of the product was measured to be 130 mM⁻¹s⁻¹, and the solubility was greater than 50 mg/mL in water.

Elemental analysis for Hydrochalarone-3 indicates a product with 12 pendent groups having the formula C₁₆₄H₁₉₂O₄₈N₁₃Gd₃ (FIG. 14). It is noted that in elemental analysis of TRIMETASPHERE®s, incomplete combustion of the cage is a persistent problem. For example, samples of pure, unfunctionalized materials, such as Sc₃N@C₈₀, routinely result in low carbon analysis. This is undoubtedly due to the high stability of these materials, as reflected in the TGA.

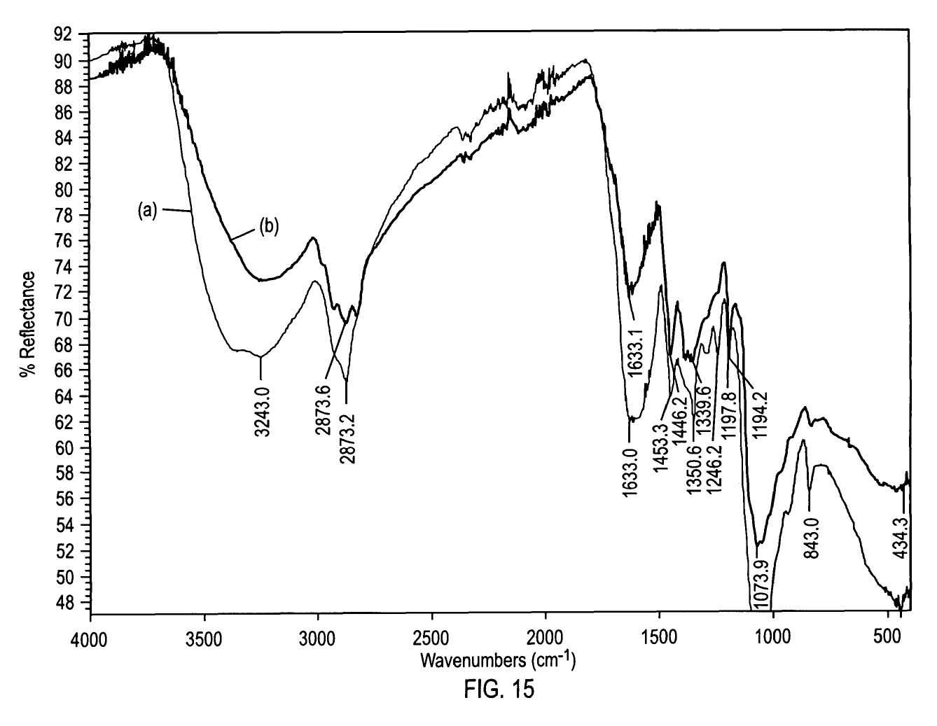

FIGS. 15(a) and 15(b) are IR spectra of C₇₀-MEG and Hydrochalarone-3 (low peroxide preparation), which show the peak at about 1452 cm⁻¹ associated with hydroxylamines.

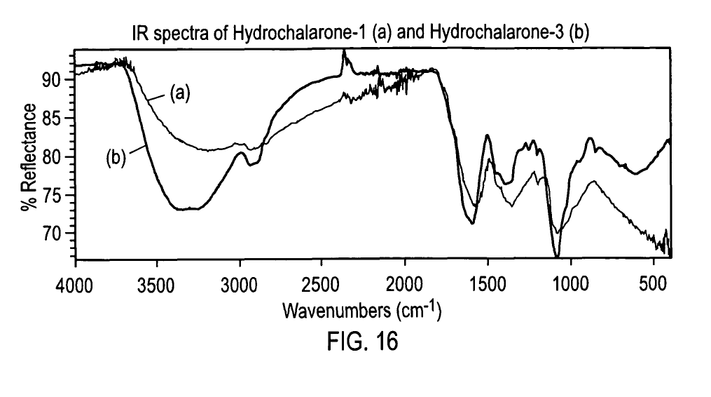

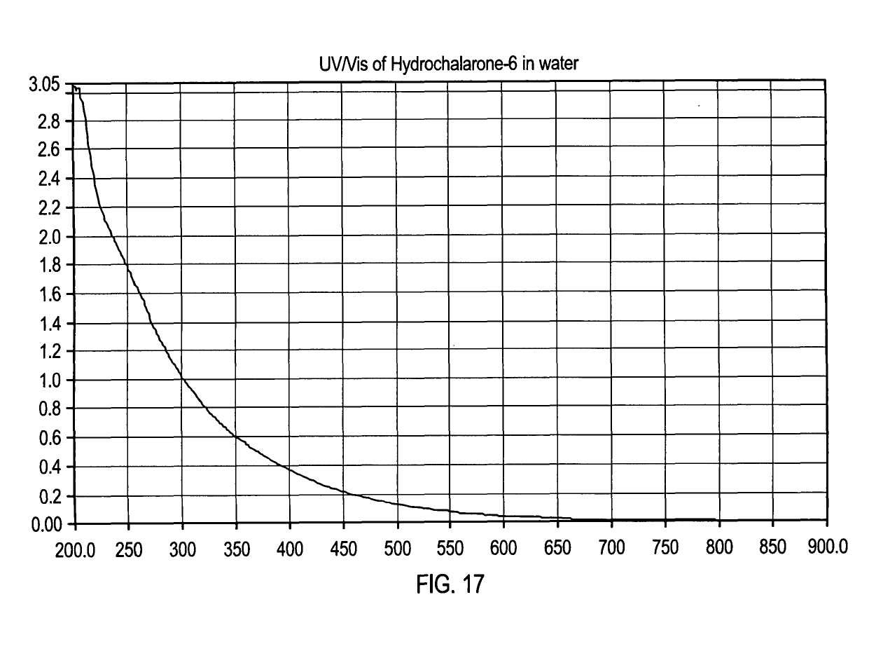

Further, Hydrochalarone-1 and Hydrochalarone-6 can be prepared in the same manner. FIGS. 16(a) and 16(b) are IR spectra of Hydrochalarone-1 and Hydrochalarone-3. FIG. 17 is UV/Vis of Hydrochalarone-6 in water. FIGS. 18(a) and 18(b) are IR spectra of GdTMS-decylamine and Hydrochalarone-1 showing C—O stretch (at about 1100 cm⁻¹) in a non-ether amine product. It is noted that GdTMS-decylamine is ether soluble while Hydrochalarone-1 is water soluble.

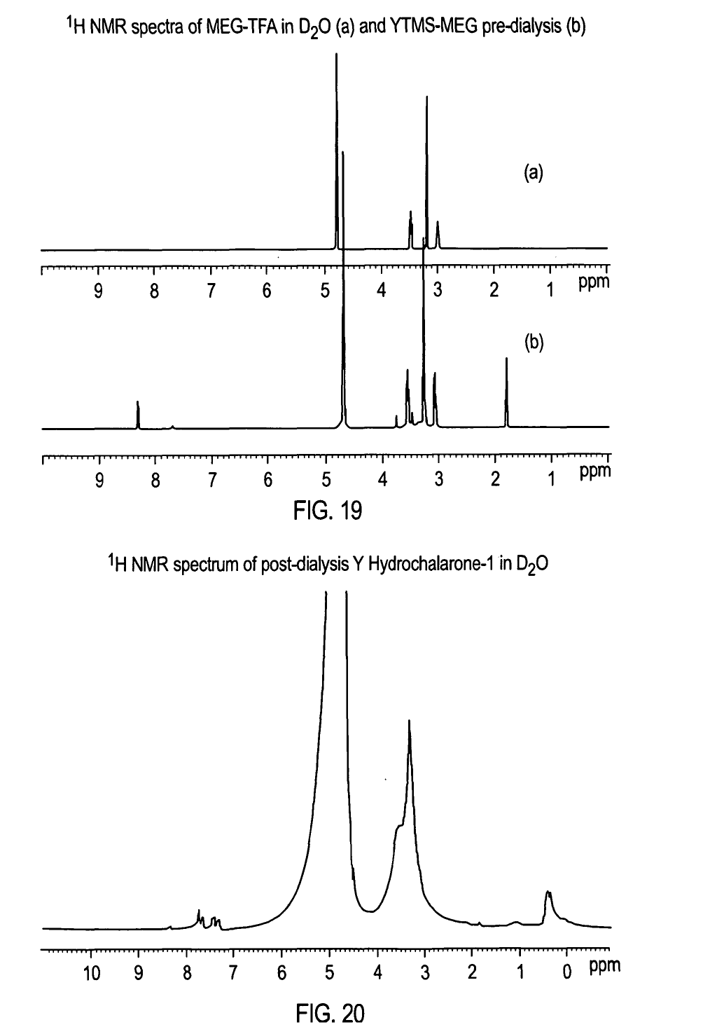

FIGS. 19(a) and (b) are ¹H NMR spectra of MEG-TFA in D₂O and YTMS-MEG pre-dialysis showing the presence of MEG-H⁺ with YTMS-MEG in the reaction mixture. FIG. 20 is a ¹H NMR spectrum of post-dialysis Y Hydrochalarone-1 in D₂O wherein MEG-H^{P} has been removed by dialysis.

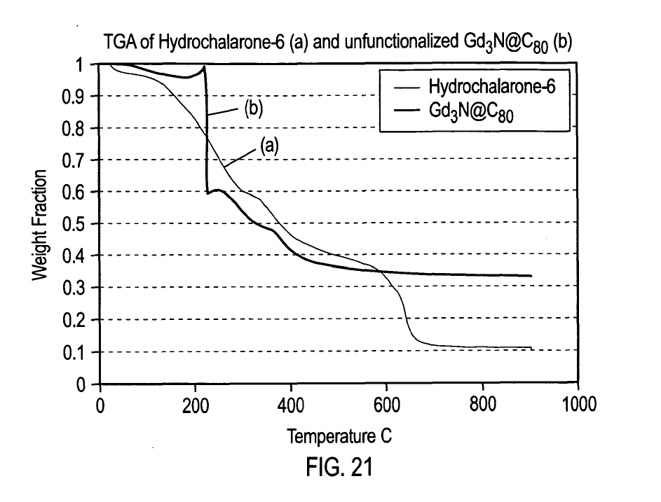

FIGS. 21(a) and (b) are TGA of Hydrochalarone-6 and unfunctionalized Gd₃N@C₈₀, which show weight loss vs. temperature. FIG. 21(b) shows that the unfunctionalized TMS starts to decompose around 200° C. and is completely decomposed at 400° C., while the Hydrochalarone derivative cage doesn't decompose until around 600° C. The rapid weight loss at around 600° C. is believed to due to the decomposition of the fullerene cage, based on the mass before the weight loss relative to the final mass, which is Gd₂O₃.

### Example 4

### Reaction of Hydrochalarone-3 in Water

Hydrochalarone-3 (2 mg) was dissolved in water (1 mL). To this solution were added NH₂(CH₂CH₂O)₃CH₃ (41 mg) and H₂O₂ (23 mg). The mixture was allowed to react for 20 hours and the color thereof faded. Measurement of water proton relaxation parameter T₁ (Maran Ultra relaxometer) showed a change from 256 ms to 395 ms, indicating an increase in the number of substitution on the carbon cage.

### Example 5

### Solid Phase Reaction with Fullerenes

Silica modified to have 3-aminopropyl groups appended to its surface was suspended in xylene and treated with butanone peroxide. The solid was then filtered and washed with xylene. With free peroxide thus removed, the solid was added to a solution containing a mixture of fullerenes that included C₆₀, C₇₀, and metalloendohedral fullerenes of the general formula M₃N@C₈₀. Within one hour, the C₆₀ and C₇₀ were removed from solution. This experiment demonstrates that the reaction takes place using a reactive intermediate generated by reaction of the amine and peroxide rather than direct reaction of the amine with the fullerene.

While various embodiments have been described with reference to specific embodiments, variations and modifications may be made without departing from the spirit and the scope of the invention. Such variations and modifications are to be considered within the purview and scope of the invention as defined by the appended claims.

All of the above-mentioned references are herein incorporated by reference in their entirety to the same extent as if each individual reference was specifically and individually indicated to be incorporated herein by reference in its entirety.

What is claimed is:

1. A process for producing a fullerene derivative, comprising reacting a fullerene compound with a primary amine in the presence of a peroxide, to form the fullerene derivative.

2. The process of claim 1, wherein the fullerene compound comprises a fullerene having from about 60 to about 200 carbon atoms.

3. The process of claim 1, wherein the fullerene compound comprises an endohedral metallofullerene compound.

4. The process of claim 3, wherein the endohedral metallofullerene compound comprises a compound represented by formula Gd₃N@Cₘ, wherein Cₘ represents a fullerene molecule having m carbon atoms, m represents an even integer from about 60 to about 200.

5. The process of claim 4, wherein m represents 60, 70 or 80.

6. The process of claim 1, wherein the primary amine comprises a compound represented by formula RNH₂, wherein R represents H, NH₂(CH₂)ₐCH₃ and NH₂(CH₂CH₂O)ₐCH₃ wherein a is an integer of 1 to 20.

7. The process of claim 1, wherein the primary amine comprises a compound represented by formula RNH₂, wherein R represents H, ethyl, propyl, hexyl, dodecyl, —CH(CH₂OCH₃)₂, —(CH₂)₄—NH₂, —(CH₂)₁₀—NHBoc, —(CH₂)₂—COOH, —CH(COOCH₂CH₃)(CH₂)₂Ph, —CH₂-18-crown-6, -glyme-OCH₃, -glyme-NHBoc, -triglyme-OCH₃, -triglyme-NHBoc, -hexaglyme-OCH₃ or -hexaglyme-NHBoc.

8. The process of claim 1, wherein the peroxide comprises butanone peroxide, cumene hydroperoxide, or hydrogen peroxide.

9. The process of claim 8, wherein the peroxide comprises butanone peroxide.

10. The process of claim 1, wherein the reacting is conducted in the presence of a solvent selected from the group consisting of benzene, toluene, xylene, dichlorobenzene, water and mixtures thereof.

11. The process of claim 10, wherein the solvent comprises xylene.

12. The process of claim 1, wherein the reacting is carried out in the presence of water, wherein the peroxide comprises hydrogen peroxide.

13. The process of claim 1, wherein the reacting is conducted in the presence of a solvent, and the concentration of the fullerene ranges from about 0.25 mg/mL to about 5.0 mg/mL, based on the volume of the solvent.

14. The process of claim 1, wherein the fullerene:amine molar ratio ranges from about 1:50 to about 1:750.

15. The process of claim 1, wherein amine:peroxide molar ratio ranges from about 1:1 to about 1:5.

16. The process of claim 1, wherein the fullerene:amine:peroxide molar ratio is about 1:500:1000.

17. The process of claim 1, wherein the reacting is carried out at a temperature ranging from ambient temperature to about 80° C.

18. The process of claim 1, further comprising isolating the formed fullerene derivative.

19. The process of claim 1, wherein the fullerene derivative has a water solubility of greater than about 40 mg/mL.

20. The process of claim 1, wherein the fullerene derivative has an ether solubility of about 10 mg/mL or more.

21. The process of claim 3, wherein the fullerene derivative has a relaxivity of greater than about 40 mM⁻¹s⁻¹.

22. A fullerene derivative prepared by the process of claim 1.
